(19) United States
(12) Patent Application Publication
(10) Pub. No.: US 2014/0296891 A1
(43) Pub. Date: Oct. 2, 2014

(54) FLUID EJECTION DEVICE AND MEDICAL APPARATUS

(71) Applicant: SEIKO EPSON CORPORATION, Tokyo (JP)

(72) Inventors: Hideki KOJIMA, Matsumoto-shi (JP); Hirokazu SEKINO, Chino-shi (JP)

(73) Assignee: SEIKO EPSON CORPORATION, Tokyo (JP)

(21) Appl. No.: 14/226,960

(22) Filed: Mar. 27, 2014

(30) Foreign Application Priority Data
Mar. 28, 2013 (JP) 2013-067716

(51) Int. Cl. A61B 17/3203

(52) U.S. Cl. CPC A61B 17/3203; A61B 2560/02
USPC 606/167; 239/589

(57) ABSTRACT

A fluid ejection device which ejects a fluid includes: an ejection tube containing a conductive material and having an opening for the fluid; a fluid chamber communicating with the ejection tube; an actuator which operates on receiving supply of electric energy, thereby changing pressure of the fluid in the fluid chamber; and an insulating member which is made of an insulating material and electrically insulates a member forming the fluid chamber and the ejection tube from each other.

This application claims the benefit of Japanese Patent Application No. 2013-67716, filed on Mar. 28, 2013. The content of the aforementioned application is incorporated herein by reference in its entirety.

### BACKGROUND

1. Technical Field

The present invention relates to a fluid ejection device and a medical apparatus using the fluid ejection device.

2. Related Art

As a medical apparatus which ejects a fluid to an affected part for treatment, for example, a device disclosed in JP-A-2008-82202 is known. In the fluid ejection device disclosed in JP-A-2008-82202, a piezoelectric element is driven to increase or decrease the volume of a fluid chamber, thus causing a pulsed flow (pulse flow) to be ejected from an ejection tube.

However, in some cases, during a surgical operation using a fluid ejection device, another medical apparatus (for example, an electric knife) that generates electric noise may be used. If this another medical apparatus touches the ejection tube of the fluid ejection device, electric noise can propagate to the piezoelectric element via the ejection tube.

This problem is not limited to the fluid ejection device that generates pulsation in a fluid by using the piezoelectric element but also commonly applies to fluid ejection devices in general which use an actuator that operates on receiving supply of electric energy.

Moreover, a reduction in size, a reduction in cost, resource saving, easier manufacturing, improvement in usability and the like are demanded of the traditional fluid ejection devices.

### SUMMARY

An advantage of some aspects of the invention is to solve at least a problems described above, and an aspect of the invention can be implemented as the following aspects.

(1) An aspect of the invention provides a fluid ejection device which ejects a fluid. The fluid ejection device includes: an ejection tube containing a conductive material and having an opening for the fluid; a fluid chamber communicating with the ejection tube; an actuator which operates on receiving supply of electric energy, thereby changing pressure of the fluid in the fluid chamber; and an insulating member which is made of an insulating material and electrically insulates a member forming the fluid chamber and the ejection tube from each other. According to the fluid ejection device of this aspect, since the member forming the fluid chamber and the ejection tube are electrically insulated from each other, even if electric noise is applied to the ejection tube, propagation of the electric noise to the actuator via the member forming the fluid chamber can be restrained.

(2) In the fluid ejection device of the aspect described above, the insulating member may include an insulating channel which communicates with the fluid chamber, and the ejection tube may communicate with the insulating channel. According to the fluid ejection device of this aspect, the member forming the fluid chamber and the ejection tube can be electrically insulated from each other with a simple configuration.

(3) In the fluid ejection device of the aspect described above, the insulating channel may be a tubular insulating tube, and the fluid ejection device may further include a reinforcing member on an outer periphery of the insulating tube. According to the fluid ejection device of this aspect, since the reinforcing member is provided on the outer periphery of the insulating tube, the insulating tube can be reinforced and flexure or the like of the insulating tube can be restrained.

(4) In the fluid ejection device of the aspect described above, an outlet tube communicating with the fluid chamber and the insulating tube may be provided between the fluid chamber and the insulating tube, the reinforcing member may have a length that is longer than a distance between a rear end of the ejection tube and a forward end of the outlet tube, the insulating tube and the ejection tube may be situated on an inner side of a forward end part of the reinforcing member, and the insulating tube and the outlet tube may be situated on an inner side of a rear end part of the reinforcing member. According to the fluid ejection device of this aspect, the reinforcing member, the insulating tube and the ejection tube overlap with each other in the forward end part of the reinforcing member, and the reinforcing member, the insulating tube and the outlet tube overlap with each other in the rear end part of the reinforcing member. Therefore, the strength in the connection part between the insulating tube and the ejection tube and in the connection part between the insulating tube and the outlet tube can be increased.

(5) In the fluid ejection device of the aspect described above, the reinforcing member may have a length that is shorter than the insulating tube, and to outer periphery of a forward end part of the insulating tube and an outer periphery of a rear end part of the insulating tube may be exposed from the reinforcing member. According to the fluid ejection device of this aspect, the outer periphery of the forward end part of the insulating tube and the outer periphery of the rear end part of the insulating tube are exposed from the reinforcing member. Therefore, propagation of electric noise to the reinforcing member from a portion that is not covered with the insulating tube, of the outer periphery of the ejection tube, can be restrained, and propagation of electric noise from the reinforcing member to a portion that is not covered with the insulating tube, of the outer periphery of the outlet tube, can be restrained.

(6) In the fluid ejection device of the aspect described above, the reinforcing member may be made of a metal, and a portion where the reinforcing member, the insulating tube and the ejection tube overlap with each other, and a portion where the reinforcing member, the insulating tube and the outlet tube overlap with each other may be swaged from outside and thereby fixed. According to the fluid ejection device of this aspect, the reinforcing member, the insulating tube and the ejection tube can be firmly fixed together by swaging. Also, assembling time can be reduced, compared with the case where other fixing methods such as adhering are used.

(7) In the fluid ejection device of the aspect described above, the reinforcing member may be a tubular member. According to the fluid ejection device of this aspect, since the reinforcing member is a tubular member, displacement of the insulating tube in the direction of diameter when a pulsed flow passes through the insulating tube can be restrained, and a pressure change of the pulsed flow can be transmitted directly to the ejection tube.

(8) The fluid ejection device of the aspect described above may further include an insulating casing in which the member forming the fluid chamber is housed, and a connection part between the insulating channel and the member forming the fluid chamber may be situated inside the casing. According to the fluid ejection device of this aspect, since the connection part between the insulating channel and the member forming the fluid chamber is situated inside the casing, application of electric noise from the connection part can be restrained.

(9) In the fluid ejection device of the aspect described above, of an inner periphery of the insulating channel, the inner periphery at a part contacting the fluid may have a shape and size that is substantially the same as a shape and size of an inner periphery of the ejection tube. According to the fluid ejection device of this aspect, since there is no step in the channel at the connection part between the insulating channel and the ejection tube, staying of air bubbles contained in the fluid at the step and an increase in channel resistance can be restrained.

(10) In the fluid ejection device of the aspect described above, the ejection tube may be made of a metal, and the insulating member may be made of a resin. According to the fluid ejection device of this aspect, the ejection tube that cannot be easily deformed can be easily realized and the insulating member can be realized at a low cast.

(11) In the fluid ejection device of the aspect described above, the actuator may include a piezoelectric element. According to the fluid ejection device of this aspect, the pressure of the fluid in the fluid chamber can be controlled with precision.

(12) Another aspect of the invention provides a medical apparatus using the fluid ejection device of the aspect described above. According to this aspect, a highly reliable medical apparatus can be provided.

Not all of the plural components provided in each of the above aspects of the invention are essential. In order to solve a part or all of the foregoing problems, or in order to achieve a part or all of the advantages described herein, a part of the plural components can be changed, deleted, replaced with another new component, or partly deleted in a limited context. Also, in order to solve a part or all of the foregoing problems, or in order to achieve a part or all of the advantages described herein, a part or all of the technical features included in one aspect of the invention can be combined with a part or all of the technical features of another aspect of the invention, to form a different aspect of the invention.

For example, an aspect of the invention can be implemented as a device having one or more of the following four components: an ejection tube, a fluid chamber, an actuator, and an insulating member. That is, this device may or may not have an ejection tube. Similarly, the device may or may not have a fluid chamber. Also, the device may or may not have an actuator. The device may or may not have an insulating member. The ejection tube may be made of, for example, a conductive material and may be formed as an ejection tube having an opening for ejecting the fluid. The fluid chamber may be formed, for example, as a fluid chamber which communicates with the ejection tube. The actuator may be formed, for example, as an actuator which operates on receiving supply of electric energy and thereby changing the pressure of the fluid in the fluid chamber. The insulating member may be made of, for example, an insulating material and may be formed as an insulating member which electrically insulates the member forming the fluid chamber and the ejection tube from each other. Such a device can be implemented, for example as a fluid ejection device which ejects a fluid but can also be implemented as another device than the fluid ejection device. According to such an aspect, at least one of the various problems such as a reduction in the size of the device, a reduction in cost, resource saving, easier manufacturing, and improvement in usability can be solved. A part of all of the technical features of each aspect of the foregoing fluid ejection device can be applied to this device.

The invention can also be implemented in various other aspects than the device. For example, the invention can be implemented in such aspects as a method for ejecting a fluid and a method for manufacturing a fluid ejection device.

### BRIEF DESCRIPTION OF THE DRAWINGS

The invention will be described with reference to the accompanying drawings, wherein like numbers reference like elements.

FIG. 1 is an explanatory view showing the configuration of a fluid ejection device as an embodiment of the invention.

FIG. 2 is an enlarged cross-sectional view showing a part of the inner configuration of a handpiece.

FIG. 3 is an enlarged cross-sectional view showing a part of the inner configuration of a handpiece as a second embodiment.

FIG. 4 is an enlarged cross-sectional view showing a part of the inner configuration of a handpiece as a third embodiment.

### DESCRIPTION OF EXEMPLARY EMBODIMENTS

Now, embodiments of the invention will be described, using first to third embodiments and modifications.

### A. First Embodiment

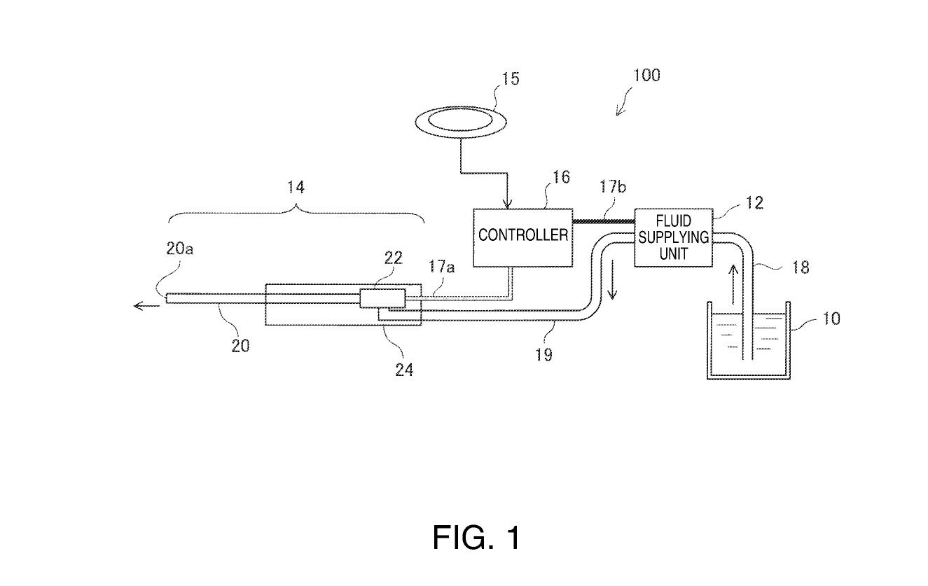

FIG. 1 is an explanatory view showing the configuration of a fluid ejection device 100 as an embodiment of the invention. The fluid ejection device 100 of this embodiment is a medical apparatus used in a medical institution and has the function of a surgical knife which ejects a fluid to an affected part and thereby incises or excises the affect part.

The fluid ejection device 100 has a fluid container 10, a fluid supplying unit 12, a handpiece 14, a foot switch 15, and a controller 16. The fluid container 10 and the fluid supplying unit 12 are connected to each other by a connection tube 18. The fluid supplying unit 12 and the handpiece 14 are connected to each other by a connection tube 19. In this embodiment, the connection tubes 18, 19 are made of an insulating resin.

The fluid container 10 houses a physiological saline solution as a fluid to be supplied to the handpiece 14. However, the fluid container 10 may house another fluid that is harmless when ejected to an affected part, for example, pure water, a drug solution or the like, instead of the physiological saline solution.

The fluid supplying unit 12 supplies the fluid housed in the fluid container 10 to the handpiece 14 made of a synthetic resin, via the connection tubes 18, 19. In this embodiment, a pump is used as the fluid supplying unit 12.

The handpiece 14 is an instrument which the operator holds in the hand and operates. The handpiece 14 has a fluid ejection tube 20, a pulsation generator 22, and a casing 24. The handpiece 14 ejects, at a high speed, the fluid in which pulsation is generated (pulsed flow) from an opening 20a at the forward end of the fluid ejection tube 20. The operator applies the fluid ejected from the handpiece 14 to an affected part of the patient and thus incises or excises the affected part. The expression “to eject the fluid in which pulsation is generated (pulsed flow)” means to eject the fluid in the state where the flow rate or flow speed of the ejected fluid varies. Therefore, this expression includes intermittent ejection in which fluid ejection and stoppage are repeated.

In this embodiment, the fluid ejection tube 20 is made of stainless steel. However, the fluid ejection tube 20 may also be made of other metals such as brass.

The controller 16 applies a drive voltage to the pulsation generator 22 via a voltage application cable 17a and also controls the fluid supplying unit 12 via a control cable 17b, thereby controlling the flow rate of the fluid supplied to the pulsation generator 22.

The foot switch 15 which the operator operates with the foot is connected to the controller 16. As the operator turns on the foot switch 15, the controller 16 applies the drive voltage to the pulsation generator 22 and the handpiece 14 ejects the fluid in which pulsation is generated (pulsed flow) at a high speed from the opening 20a at the forward end of the fluid ejection tube 20.

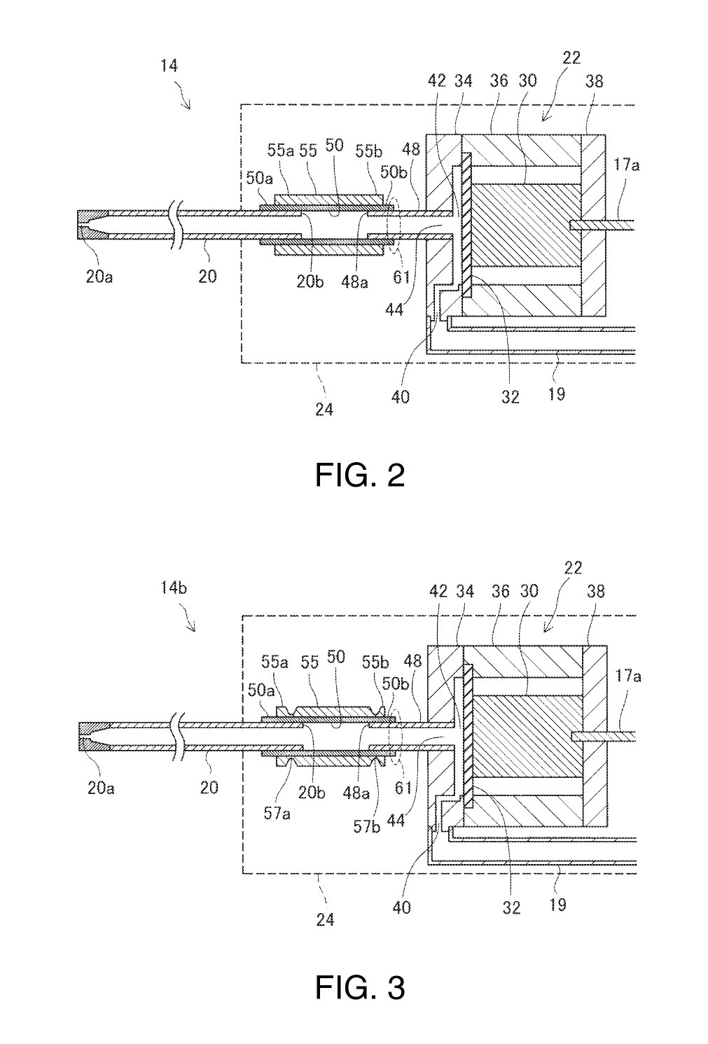

FIG. 2 is an enlarged cross-sectional view showing a part of the inner configuration of the handpiece 14. Inside the casing 24 of the handpiece 14, the pulsation generator 22 which generates pulsation in the fluid supplied from the fluid supplying unit 12 is provided. The pulsation generator 22 has a piezoelectric element 30, a diaphragm 32, a first case 34, a second case 36, and a third case 38.

Inside the pulsation generator 22, an inlet channel 40, a fluid chamber 42, and an outlet channel 44 are formed as a channel through which the fluid supplied from the fluid supplying unit 12 passes. In this embodiment, the inlet channel 40 and the outlet channel 44 are formed in the first case 34. The fluid chamber 42 is formed between the first case 34 and the diaphragm 32. An outlet tube 48 communicating with the fluid chamber 42 is connected to the outlet channel 44. Also, in this embodiment, the cases 34, 36, 38 and the outlet tube 48 are made of a metal material. The casing 24 is made of a resin which is an insulating material.

The diaphragm 32 is a disc-shaped thin metal plate and an outer peripheral part thereof is held and fixed between the first case 34 and the second case 36.

The piezoelectric element 30 is an actuator which operates on receiving a drive voltage applied from the controller 16. The piezoelectric element 30 changes the volume of the fluid chamber 42 formed between the diaphragm 32 and the first case 34, thereby changing the pressure of the fluid in the fluid chamber 42. In this embodiment, the piezoelectric element 30 is a multilayer piezoelectric element, with one end thereof fixed to the diaphragm 32 and the other end thereof fixed to the third case 38.

When the drive voltage applied to the piezoelectric element 30 increases, the piezoelectric element 30 expands. The diaphragm 32 is pushed by the piezoelectric element 30 and flexes toward the fluid chamber 42. As the diaphragm 32 flexes toward the fluid chamber 42, the volume of the fluid chamber 42 decreases and the fluid in the fluid chamber 42 is extruded from the fluid chamber 42. In this embodiment, the inner diameter of the outlet channel 44 is greater than the inner diameter of the inlet channel 40. Therefore, the inertance of the inlet channel 40 is greater than the inertance of the outlet channel 44. This restrains backflow of the fluid in the fluid chamber 42 to the inlet channel 40, and the fluid is extruded from the fluid chamber 42 via the outlet channel 44.

Meanwhile, when the drive voltage applied to the piezoelectric element 30 decreases, the piezoelectric element 30 contracts and the volume of the fluid chamber 42 increases. Thus, the fluid is supplied into the fluid chamber 42.

Since the controller 16 periodically changes the drive voltage applied to the piezoelectric element 30, an expansion and contraction of the volume of the fluid chamber 42 is repeated, generating pulsation in the fluid. The fluid extruded from the fluid chamber 42 is ejected from the nozzle 20a (opening 20a) at the forward end of the fluid ejection tube 20.

In this embodiment, an insulating tube 50 made of an insulating material is provided between the outlet tube 48 and the fluid ejection tube 20. The insulating tube 50 functions as an insulating member which electrically insulates the first case 34 as a member forming the fluid chamber 42 and the fluid ejection tube 20 from each other. Therefore, according to this embodiment, even if electric noise is applied to the fluid ejection tube 20 made of stainless steel, propagation of the electric noise to the piezoelectric element 30 via the first case 34 can be restrained. That is, according to this embodiment, both high strength of the metal and insulation can be achieved.

In this embodiment, the insulating tube 50 is made of polyethylene. However, the insulating tube 50 may be made of another insulating material. For example, the insulating tube 50 may be made of another resin such as polypropylene.

Moreover, in this embodiment, a reinforcing tube 55 made of a metal is formed on the outer periphery of the insulating tube 50, as a reinforcing member to increase the rigidity of the insulating tube 50. Therefore, flexure of the insulating tube 50 can be restrained and the position of the nozzle 20a of the fluid ejection tube 20 can be fixed.

In this embodiment, the reinforcing tube 55 is made of stainless steel. However, the reinforcing tube 55 may be made of another metal such as brass.

Moreover, in this embodiment, since the tubular reinforcing tube 55 is used as a reinforcing member of the insulating tube 50, displacement of the insulating tube 50 in the direction of diameter when a pulsed flow passes through the insulating tube 50 can be restrained, and a pressure change of the pulsed flow can be propagated directly to the fluid ejection tube 20.

Also, in this embodiment, the length of the reinforcing tube 55 is longer than the distance between a rear end 20b of the fluid ejection tube 20 and a forward end 48a of the outlet tube 48. The insulating tube 50 and the fluid ejection tube 20 are situated on the inner side of a forward end portion 55a of the reinforcing tube 55. The insulating tube 50 and the outlet tube 48 are situated on the inner side of a rear end portion 55b of the reinforcing tube 55. Therefore, in this embodiment, the reinforcing tube 55, the insulating tube 50 and the fluid ejection tube 20 overlap with each other in the forward end portion 55a of the reinforcing tube 55, and the reinforcing tube 55, the insulating tube 50 and the outlet tube 48 overlap with each other in the rear end portion 55b of the reinforcing tube 55. Therefore, according to this embodiment, strength in the connection part between the insulating tube 50 and the fluid ejection tube 20 and in the connection part between the insulating tube 50 and the outlet tube 48 can be increased. The portion where the reinforcing tube 55, the insulating tube 50 and the fluid ejection tube overlap with each other, and the portion where the reinforcing tube 55, the insulating tube 50 and the outlet tube 48 overlap with each other are fixed, for example, with an adhesive or the like.

Moreover, in this embodiment, the length of the reinforcing tube 55 is shorter than the length of the insulating tube 50, and the outer periphery of a forward end portion 50a of the insulating tube 50 and the outer periphery of a rear end portion 50b of the insulating tube 50 are exposed from the reinforcing tube 55. Therefore, according to this embodiment, propagation of electric noise to the reinforcing tube 55 from a portion that is not covered with the insulating tube 50, of the outer periphery of the fluid ejection tube 20, can be restrained, and propagation of electric noise from the reinforcing tube 55 to a portion that is not covered with the insulating tube, of the outer periphery of the outlet tube 48, can be restrained.

Also, in this embodiment, a connection part 61 between the insulating tube 50 and the outlet tube 48 is situated inside the casing 24. Therefore, according to this embodiment, application of electric noise from the connection part 61 between the insulating tube 50 and the outlet tube 48 can be restrained.

In this way, in the first embodiment, since the insulating tube 50 is provided between the fluid ejection tube 20 and the outlet tube 48, even if electric noise is applied to the fluid ejection tube 20, propagation of the electric noise to the piezoelectric element 30 can be restrained.

### B. Second Embodiment

FIG. 3 is an enlarged cross-sectional view showing apart of the inner configuration of a handpiece 14b as a second embodiment. The second embodiment differs from the first embodiment shown in FIG. 2, only in that the portion where the reinforcing tube 55, the insulating tube 50 and the fluid ejection tube 20 overlap with each other and the portion where the reinforcing tube 55, the insulating tube 50 and the outlet tube 48 overlap with each other are swaged from outside and thus fixed. The other configurations are the same as the first embodiment. In FIG. 3, swaged portions 57a, 57b which are traces of swaging are shown.

According to this second embodiment, similar effects to the first embodiment can be achieved. With the swaging, the reinforcing tube 55, the insulating tube 50 and the fluid ejection tube 20 can be fixed together firmly and the reinforcing tube 55, the insulating tube 50 and the outlet tube 48 can be fixed together firmly. Therefore, cases where adhesiveness is insufficient due to the resin selected as the material of the insulating tube 50 can be dealt with, and an adhesive or the like to fix these members together can be eliminated. Therefore, the assembling time can be reduced. However, these members may also be fixed together by using both swaging and an adhesive.

### C. Third Embodiment

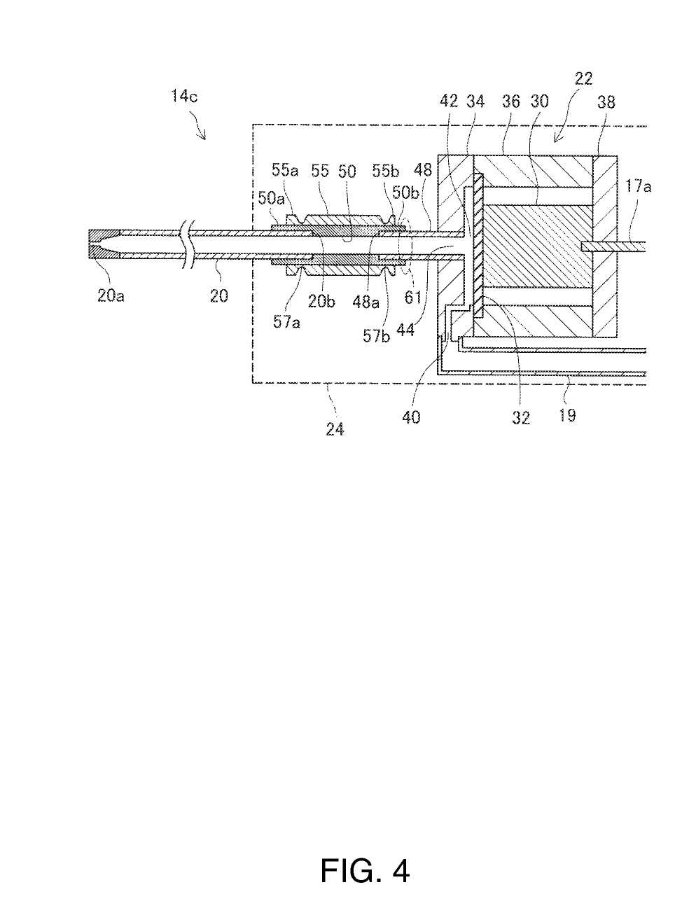

FIG. 4 is an enlarged cross-sectional view showing a part of the inner configuration of a handpiece 14c as a third embodiment. The third embodiment differs from the second embodiment shown in FIG. 3, in that the shape and size of the inner periphery of the portion contacting the fluid, of the inner periphery of the insulating tube 50, is substantially the same as the shape and size of the inner periphery of the fluid ejection tube 20. The other configurations are the same as the second embodiment.

Specifically, in this embodiment, the portion contacting the fluid, of the inner periphery of the insulating tube 50, has a smaller inner diameter. The inner diameter of the insulating tube 50 at the portion contacting the fluid is substantially equal to the inner diameter of the fluid ejection tube 20. Therefore, as there is no step in the channel in the connection part between the insulating tube 50 and the fluid ejection tube 20, staying of air bubbles contained in the fluid at the step can be restrained. As a result, weakening of the propagation of the pressure in the channel due to the air bubbles can be restrained. Also, an increase in channel resistance due to the presence of the step can be restrained.

Moreover, in this embodiment, the shape and size of the inner periphery at the portion contacting the fluid, of the inner periphery of the insulating tube 50, is substantially the same as the shape and size of the inner periphery of the outlet tube 48. Specifically, in this embodiment, the inner diameter of the insulating tube 50 at the portion contacting the fluid is substantially equal to the inner diameter of the outlet tube 48. Therefore, as there is no step in the channel at the connection part between the insulating tube 50 and the outlet tube 48, staying of air bubbles contained in the fluid at the step and an increase in channel resistance due to the presence of the step can be restrained.

Thus, according to the third embodiment, similar effects to the first embodiment and the second embodiment can be achieved. Also, since there is no step in the channel, catching of air bubbles contained in the fluid at the step and an increase in channel resistance due to the presence of the step can be restrained.

### D. Modifications

The invention is not limited to the above embodiments and can be carried out in various forms without departing from the scope of the invention. For example, the following modifications can be made.

### Modification 1

In the embodiments, the insulating tube 50 forming a part of the channel is employed as an insulating member to electrically insulate the member forming the fluid chamber 42 and the fluid ejection tube 20 from each other. However, as a modification, an insulating member that does not form the channel may be employed as an insulating member to electrically insulate the member forming the fluid chamber 42 and the fluid ejection tube 20 from each other. For example, an insulating member may be provided between the outlet tube 48 and the first case 34.

### Modification 2

In the embodiments, the outlet tube 48 is provided between the insulating tube 50 and the first case 34. However, as a modification, the outlet tube 48 may be omitted, and the insulating tube 50 may be directly connected to the first case 34.

### Modification 3

In the embodiments, the outlet tube 48 and the fluid ejection tube 20 are inserted on the inner periphery of the insulating tube 50. However, as a modification, the insulating tube 50 may be inserted on the inner peripheries of the outlet tube 48 and the fluid ejection tube 20.

### Modification 4

In the embodiments, the tubular reinforcing tube 55 is employed as a reinforcing member to reinforce the insulating tube 50. However, as a modification, a reinforcing member of another shape may be employed. For example, a plate-like reinforcing member may be employed as a reinforcing member to reinforce the insulating tube 50.

### Modification 5

In the embodiments, the length of the reinforcing tube 55 is shorter than the length of the insulating tube 50. However, as a modification, the length of the reinforcing tube 55 may be the same as the length of the insulating tube 50.

### Modification 6

In the embodiments, the piezoelectric element 30 is used as an actuator that operates on receiving supply of electric energy. However, as a modification, another type of actuator than the piezoelectric element 30 may be used. For example, a solenoid actuator, an actuator using a magnetostrictive element, or the like may be used as an actuator that changes the pressure of the fluid in the fluid chamber 42.

### Modification 7

In the embodiments, the fluid ejection device 100 is used as a medical apparatus. However, as a modification, the fluid ejection device 100 may be used as another apparatus than the medical apparatus. For example, the fluid ejection device 100 may be used as a cleaning device which ejects a fluid to a target object and thus removes stains from the target object, or a drawing device which draws letters and pictures with the ejected fluid.

In the embodiments, a liquid is used as a fluid ejected from the fluid ejection device 100. However, as a modification, a gas may be used as a fluid ejected from the fluid ejection device 100.

The invention is not limited to the above embodiments, examples and modifications and can be realized in various configurations without departing from the scope of the invention. For example, the technical features in the embodiments, examples and modifications corresponding to the technical features in the respective embodiments described in the summary of the invention can be suitably replaced or combined in order to solve a part or all of the foregoing problems or in order to achieve a part or all of the foregoing advantages. Also, the technical features can be suitably deleted unless these features are described as essential herein.

What is claimed is:

1. A fluid ejection device which ejects a fluid, comprising:
an ejection tube containing a conductive material and having an opening for the fluid;
a fluid chamber communicating with the ejection tube;
an actuator which operates on receiving supply of electric energy, thereby changing pressure of the fluid in the fluid chamber; and
an insulating member which is made of an insulating material and electrically insulates a member forming the fluid chamber and the ejection tube from each other.

2. The fluid ejection device according to claim 1, wherein the insulating member includes an insulating channel which communicates with the fluid chamber, and
the ejection tube communicates with the insulating channel.

3. The fluid ejection device according to claim 2, wherein the insulating channel is a tubular insulating tube, and
the fluid ejection device further includes a reinforcing member on an outer periphery of the insulating tube.

4. The fluid ejection device according to claim 3, wherein an outlet tube communicating with the fluid chamber and the insulating tube is provided between the fluid chamber and the insulating tube,
the reinforcing member has a length that is longer than a distance between a rear end of the ejection tube and a forward end of the outlet tube,
the insulating tube and the ejection tube are situated on an inner side of a forward end part of the reinforcing member, and
the insulating tube and the outlet tube are situated on an inner side of a rear end part of the reinforcing member.

5. The fluid ejection device according to claim 4, wherein the reinforcing member has a length that is shorter than the insulating tube, and
an outer periphery of a forward end part of the insulating tube and an outer periphery of a rear end part of the insulating tube are exposed from the reinforcing member.

6. The fluid ejection device according to claim 4, wherein the reinforcing member is made of a metal, and
a portion where the reinforcing member, the insulating tube and the ejection tube overlap with each other, and a portion where the reinforcing member, the insulating tube and the outlet tube overlap with each other are swaged from outside and thereby fixed.

7. The fluid ejection device according to claim 3, wherein the reinforcing member is a tubular member.

8. The fluid ejection device according to claim 2, further comprising an insulating casing in which the member forming the fluid chamber is housed,
wherein a connection part between the insulating channel and the member forming the fluid chamber is situated inside the casing.

9. The fluid ejection device according to claim 2, wherein, of an inner periphery of the insulating channel, the inner periphery at apart contacting the fluid may have a shape and size that is substantially the same as a shape and size of an inner periphery of the ejection tube.

10. The fluid ejection device according to claim 1, wherein the ejection tube is made of a metal, and
the insulating member is made of a resin.

11. The fluid ejection device according to claim 1, wherein the actuator includes a piezoelectric element.

12. A medical apparatus using the fluid ejection device according to claim 1.

13. A medical apparatus using the fluid ejection device according to claim 2.

14. A medical apparatus using the fluid ejection device according to claim 3.

15. A medical apparatus using the fluid ejection device according to claim 4.

16. A medical apparatus using the fluid ejection device according to claim 5.

17. A medical apparatus using the fluid ejection device according to claim 6.

18. A medical apparatus using the fluid ejection device according to claim 7.

19. A medical apparatus using the fluid ejection device according to claim 8.

20. A medical apparatus using the fluid ejection device according to claim 9.
